(19) United States
(12) Patent Application Publication
(10) Pub. No.: US 2025/0242126 A1
(43) Pub. Date: Jul. 31, 2025

(54) HIGH FLOW THERAPY AND ASSOCIATED SYSTEMS AND METHODS

(71) Applicant: VENTEC LIFE SYSTEMS, INC., Bothell, WA (US)

(72) Inventors: Samir AHMAD, San Diego, CA (US); Joseph CIPOLLONE, Mission Viejo, CA (US)

(21) Appl. No.: 18/855,626

(22) PCT Filed: Apr. 12, 2022
(86) PCT No.: PCT/US2022/071677

§ 371 Date: Oct. 9, 2024

(51) Int. Cl. A61M 16/22; A61M 16/00; A61M 16/10

(52) U.S. Cl. CPC A61M 16/22; A61M 16/024; A61M 16/1005; A61M 16/108; A61M 2016/0027; A61M 2016/0036

(57) ABSTRACT

The present technology is directed to systems and methods that reduce the amount of oxygen used during high flow therapy. For example, the present technology includes systems and methods that (1) deliver oxygen to the patient during the inhalation phase of a breath at a flow rate between 15 lpm and 60 lpm. and (2) deliver air to the patient during the exhalation phase of the breath at a flow rate between 15 lpm and 60 lpm. Accordingly, the systems and methods can maintain a high flow to the patient during both the inhalation phase and the exhalation phase, but can change the type of gas being delivered to the patient during each phase to conserve oxygen.

### TECHNICAL FIELD

The present technology is generally directed to systems and methods for providing respiratory therapy, and in particular to systems and methods for providing high flow therapy with reduced oxygen consumption.

### BACKGROUND

Standard oxygen therapy is used as the primary treatment for hypoxemic patients. Standard oxygen therapy includes administering oxygen to the patient at relatively low flows, such as up to 15 liters-per-minute (lpm), using devices such as nasal cannulas, non-rebreathing masks, and bag-valve masks. The fraction of inspired oxygen (FiO2) obtained using these devices varies with the patient's breathing pattern, peak inspiratory flow rate, delivery system, and mask characteristics. High flow oxygen therapy is an alternative to standard oxygen therapy. In high flow oxygen therapy, oxygen is warmed and humidified before being delivered to the patient. Because the oxygen is warmed and humidified, the oxygen can be delivered at higher flow rates up to 60 lpm and with FiO2 values of nearly 100%. Depending on the patient, high flow oxygen therapy can provide a number of physiological benefits including greater comfort and tolerance, more effective oxygenation under some circumstances, and breathing pattern improvements with an increase in tidal volume and decreases in respiratory rate and dyspnea.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1 is a schematic illustration of a standard system for providing high flow oxygen therapy.

FIG. 2 is a schematic illustration of a system for providing high flow therapy and configured in accordance with embodiments of the present technology.

FIG. 3 is a graph illustrating various aspects of a high flow therapy administered to a patient using the system shown in FIG. 2 and in accordance with embodiments of the present technology.

FIG. 4 is a flowchart of a method for delivering high flow therapy in accordance with embodiments of the present technology.

### DETAILED DESCRIPTION

High flow oxygen therapy is an alternative to standard oxygen and generally includes warming and or humidifying oxygen before delivering it to the patient. Because the oxygen is warmed and/or humidified, the patient can tolerate increased flows, such as between 15 lpm and 60 lpm. High flow oxygen therapy may be beneficial to certain patients who require greater volumes of oxygen delivery and/or who do not respond well to standard low flow oxygen therapy. However, because high flow oxygen therapy provides oxygen at high flows, high flow oxygen therapy consumes oxygen at a much faster rate than standard low flow oxygen therapy. Accordingly, a need exists to improve high flow oxygen therapy to retain the benefits provided by high flow therapy while minimizing the amount of oxygen used during therapy.

The present technology is directed to systems and methods that reduce the amount of oxygen used during high flow therapy. For example, the present technology includes systems and methods that (1) deliver oxygen to the patient during the inhalation phase of a breath at a flow rate between 15 lpm and 60 lpm, and (2) deliver air to the patient during the exhalation phase of the breath at a flow rate between 15 lpm and 60 lpm. In some embodiments, the delivery of oxygen is ceased during the exhalation phase and the delivery of air is ceased during the inhalation phase, although in other embodiments combinations of oxygen and air can be delivered during the inhalation phase and/or the exhalation phase such that the total flow rate is between 15 lpm and 60 lpm. Regardless, the systems and methods described herein can maintain a high flow to the patient during both the inhalation phase and the exhalation phase to provide the advantages associated with high flow therapy while also changing the type or amount of gas being delivered to the patient during each phase to conserve oxygen. Accordingly, the present technology is expected to reduce the amount of oxygen used during high flow therapy at least by reducing the amount of oxygen delivered to the patient, and thus “wasted,” during the exhalation phase. Without being bound by theory, this may reduce the cost of high flow oxygen therapy, reduce the oxygen wastage during high flow oxygen therapy, and/or prolong the life of certain high flow therapy systems. Further aspects and advantages of the devices, methods, and uses will become apparent from the ensuing description that is given by way of example only.

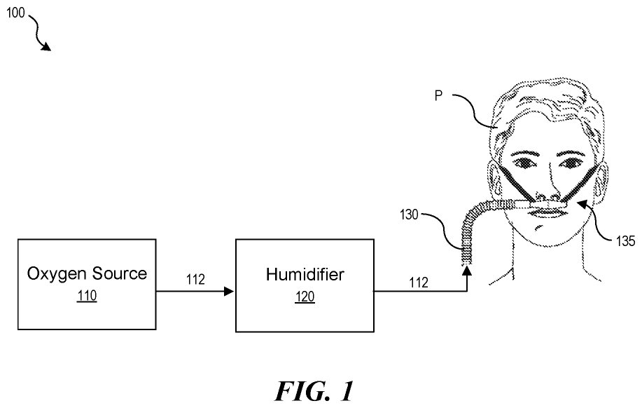

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims or examples but are not described in detail with respect to FIGS. 1-4.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%. The term “substantially” or grammatical variations thereof refers to at least about 50%, for example, 75%, 85%, 95%, or 98%.

FIG. 1 is a schematic diagram of a standard high flow therapy system 100 (“the system 100”) for delivering high flow oxygen therapy. The system 100 includes an oxygen source 110 for providing oxygen 112 to a humidifier 120, which heats and humidifies the oxygen 112. The oxygen 112 then passes through a patient circuit 130 (e.g., corrugated tubing or the like) before being delivered to a patient P via a patient connection 135, such as a nasal cannula or the like. During standard high flow therapy, oxygen 112 is delivered to the patient at a constant or substantially constant flow regardless of the breath phase. For example, oxygen 112 may be delivered at a flow rate of up to 60 lpm during both an inhalation phase and an exhalation phase of a breath. During the inhalation phase, the oxygen 112 is directed into the patient's lungs and upper airways to fill the patient's alveoli and provide an oxygenated breath to the user. During the exhalation phase, the oxygen 112 is directed into the patient's upper airways to assist with clearing carbon dioxide (CO₂) included in the patient's exhalation gases. This is advantageous because it reduces rebreathing of CO₂ during subsequent breaths. However, due to the substantially constant high flow values during both inhalation and exhalation necessary to achieve these advantages of high flow therapy, a relatively large volume of oxygen is consumed (e.g., up to 60 liters of oxygen per minute if the oxygen is being delivered at a flow rate of 60 lpm).

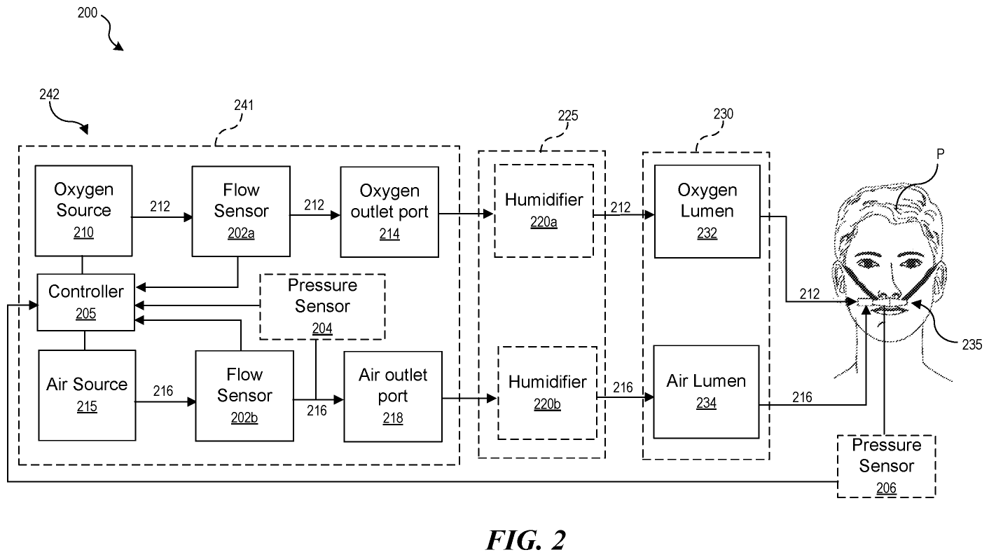

The present technology is directed to systems and methods that are expected to decrease the volume of oxygen necessary to perform high flow therapy while retaining the advantages associated with high flow therapy. FIG. 2 is a schematic diagram of a high flow therapy system 200 (“the system 200”) for delivering high flow therapy and configured in accordance with select embodiments of the present technology. The system 200 can include both an oxygen source 210 and an air source 215. The oxygen source 210 can provide oxygen 212 (e.g., concentrated oxygen that is greater than 21% O₂, such as between about 80% and about 100% O₂). The air source 215 can provide air 216 (e.g., ambient air, pressurized air, or other gases). In some embodiments, the oxygen source 210 and the air source 215 are contained within the same housing 241 such that the oxygen source 210 and the air source 215 are part of the same device 242 (e.g., a portable oxygen concentrator, a portable multi-functional ventilator, a high flow therapy device, etc.). In other embodiments, one or both of the oxygen source 210 and the air source 215 can be or form part of separate devices. For example, the oxygen source 210 can be an external oxygen source such as an oxygen tank.

The oxygen source 210 is configured to deliver the oxygen 212 to an oxygen delivery lumen 232 (also referred to simply as “the oxygen lumen 235”). In embodiments in which the oxygen source 210 is contained within the housing 241, the oxygen source 210 may deliver the oxygen 212 to the oxygen lumen 232 via an oxygen outlet port 214 in the housing 241. The oxygen lumen 232 routes the oxygen 212 to a patient connection 235, which delivers the oxygen 212 to the patient P. The oxygen lumen 232 can be any suitable oxygen delivery circuit, such as corrugated tubing or another suitable conduit (e.g., tubing extending from a nasal cannula). The patient connection 235 can be any suitable interface for delivering the oxygen 212 to the patient, such as a nasal cannula, nasal mask, mouth mask, face mask, mouthpiece, tracheal tube, tracheostomy tube or interface, or the like. In some embodiments, the oxygen lumen 232 and the patient connection 235 are a single component (e.g., a nasal cannula).

The system 200 can optionally include a first humidifier 220a positioned between or otherwise fluidly coupled between the oxygen source 210 and the oxygen lumen 232. The first humidifier 220a can therefore heat and/or humidify the oxygen 212 before it is delivered to the patient P at the patient connection 235. Although the first humidifier 220a is shown “upstream” of the oxygen lumen 232, one skilled in the art will appreciate that the system 200 can be configured such that the first humidifier 220a is positioned elsewhere, such as along a length of the oxygen lumen 232, between the oxygen lumen 232 and the patient connection 235, and/or within the housing 241 of the device 242. In some embodiments, the first humidifier 220a can be omitted and the oxygen can optionally be heated/humidified by the oxygen lumen 232.

The air source 215 is configured to deliver the air 216 to an air delivery lumen 234 (also referred to simply as “the air lumen 234”). In embodiments in which the air source 215 is contained within the housing 241, the air source 215 may deliver the air 216 to the air lumen 234 via an air outlet port 218 in the housing 241. The air lumen 234 routes the air 216 to the patient connection 235, which delivers the air 216 to the patient P. The air lumen 234 can be any suitable air delivery circuit, such as corrugated tubing or another suitable conduit (e.g., tubing extending from a nasal cannula). Of note, the air lumen 234 is fluidly isolated from the oxygen lumen 232 until at or proximate the patient connection 235 (e.g., within 16 inches, within 12 inches of, within 10 inches of, within 8 inches of, within 6 inches of, within 4 inches of, within 3 inches of, within 2 inches of, within 1 inch of, or at a distal terminus of the patient connection 235 that releases the oxygen 212 and the air 216 into the patient P). Accordingly, the oxygen 212 and the air 216 do not mix until proximate and/or at the patient connection 235. In some embodiments, the air lumen 234 and the patient connection 235 are a single component (e.g., a nasal cannula).

In some embodiments, the oxygen lumen 232 and the air lumen 234 can form a single gas delivery circuit to reduce the number of system components. For example, the system 200 can include a patient circuit 230 that includes the oxygen lumen 232 and the air lumen 234. In such embodiments, the patient circuit 230 can include a multi-lumen tubing, multi-lumen conduit, or the like that includes the oxygen lumen 232 and the air lumen 234. The oxygen lumen 232 and the air lumen 234 can have a side-by-side arrangement, in which the two lumens are simply joined together (e.g., glued, molded, tied, etc.) along their lengths. The oxygen lumen 232 and the air lumen 234 can also both be positioned within a larger conduit of the patient circuit 230. In yet other embodiments, the oxygen lumen 232 and the air lumen 234 can have a co-axial configuration, in which one of the oxygen lumen 232 and the air lumen 234 is positioned inside of the other. An example of a multi-lumen patient circuit that can be used with the present technology is described in U.S. Pat. No. 11,191,915, the disclosure of which is incorporated by reference herein in its entirety. Incorporating the oxygen lumen 232 and the air lumen 234 into a single component (e.g., the patient circuit 230) is expected to be advantageous because it reduces the number of components necessary to operate the system 200, and therefore reduces the burden on a patient during transportation and activities, and reduces the likelihood that one of the lumens gets tangled or inadvertently caught on a foreign object. However, in some embodiments, the oxygen lumen 232 and the air lumen 234 are separate or substantially separate components. In such embodiments, the oxygen lumen 232 and the air lumen 234 generally are not coupled along their respective lengths (excluding simple connections made by a patient or physician, such as connecting the oxygen lumen 232 and the air lumen 234 via a zip tie). Maintaining the oxygen lumen 232 and the air lumen 234 as separate components may be advantageous because if one of the oxygen lumen 232 or the air lumen 234 needs repair or replacement, the other of the oxygen lumen 232 or the air lumen 234 need not also be repaired or replaced.

The system 200 can optionally include a second humidifier 220b positioned between or otherwise fluidly coupled between the air source 215 and the air lumen 234. The second humidifier 220b can therefore heat and/or humidify the air 216 before it is delivered to the patient P at the patient connection 235. Although the second humidifier 220b is shown “upstream” of the air lumen 234, one skilled in the art will appreciate that the system 200 can be configured such that the second humidifier 220b is positioned elsewhere, such as along a length of the air lumen 234, between the air lumen 234 and the patient connection 235, and/or within the housing 241 of the device. In some embodiments, the second humidifier 220b is omitted and the air 216 can optionally be heated/humidified by the air lumen 234.

In some embodiments, the first humidifier 220a and the second humidifier 220b can be contained within a single humidifier assembly 225. For example, the humidifier assembly 225 can have a single housing the encloses both the first humidifier 220a and the second humidifier 220b. In such embodiments, certain components of the first humidifier 220a and the second humidifier 220b may be shared (e.g., a heater) to reduce the number of components necessary to operate the humidifier assembly 225. In embodiments in which the first humidifier 220a and the second humidifier 220b are contained within the humidifier assembly 225, the humidifier assembly 225 is nevertheless configured to keep the oxygen 212 and the air 216 fluidly isolated. The humidifier assembly 225 therefore provides two discrete flow paths: a first flow path between the oxygen source 210 and the oxygen lumen 232 to heat and/or humidify the oxygen 212, and a second flow path between the air source 215 and the air lumen 234 to heat and/or humidify the air 216. In other embodiments, the humidifier assembly 225 is omitted, and the first humidifier 220a and the second humidifier 220b are separate components. In other embodiments, the system 200 only includes the first humidifier 220a, and the second humidifier 220b is omitted. In such embodiments, the air 216 may not be heated or humidified before being delivered to the patient P. In yet other embodiments, the humidifier assembly 225 is omitted and the oxygen lumen 232 and/or the air lumen 234 heat/humidify the concentrated oxygen 212 and the air 216, respectively.

In some embodiments, the oxygen lumen 232 and the air lumen 234 can be fluidly joined at or proximate the patient connection 235 such that oxygen 212 and air 216 flow along a common portion of the patient circuit 230 at its distalmost portion. In such embodiments, a humidifier (not shown) can be positioned downstream of the junction between the oxygen lumen 232 and the air lumen 234 along the common portion, such that a single humidifier can humidify the air 216 received via the air lumen 234 and the oxygen 212 received via the oxygen lumen 232. However, even in embodiments in which the air lumen 234 and the oxygen lumen 232 merge into a common portion, the oxygen 212 and the air 216 are nevertheless fluidly isolated along substantially the entire length of the patient circuit 230, mixing just before entering the humidifier and being delivered to the patient P.

Returning to the embodiment illustrated in FIG. 2, the system 200 further includes a first flow sensor 202a configured to measure the flow of oxygen 212 discharged from the oxygen source 210, and a second flow sensor 202b configured to measure the flow of air 216 discharged from the air source 215 (collectively referred to as “the flow sensors 202”). The flow sensors 202 can be positioned within the housing 241 proximate the oxygen source 210 and the air source 215, or along the oxygen lumen 232 and the air lumen 234. As described below, the flow sensors 202 can be configured to measure flow during therapy. The measured flow can be used as part of an automated control mechanism for the system 200 and/or can be displayed to the patient or other user (e.g., via display screen; not shown).

The system 200 can further include one or more pressure sensors configured to measure a pressure within one or more portions of the system 200. For example, the system 200 can include a first pressure sensor 204 positioned within the housing 241, such as between the air source 215 and the air outlet port 218. Alternatively or additionally, the system 200 can include a second pressure sensor 206 positioned within or proximate the patient connection 235 and/or the patient circuit 230. As described in greater detail below, the pressure sensors can sense a change in pressure that indicates a change of phase in the patient's breath.

The system 200 further includes a controller 205 for controlling operation of the system 200. The controller 205 is configured to receive inputs from one or more of the flow sensors 202, the first pressure sensor 204, and/or the second pressure sensor 206. The controller 205 is further configured to control the oxygen source 210 and the air source 215, e.g., based on the inputs received from the flow sensors 202, the first pressure sensor 204, and/or the second pressure sensor 206. For example, based on the inputs received from the flow sensors 202, the controller 205 may direct the oxygen source 210 and/or the air source 215 to maintain a given flow, control the oxygen source 210 or the air source 215 to increase flow, control the oxygen source 210 or the air source 215 to decrease flow, etc. Based on the inputs received from the first pressure sensor 204 and/or the second pressure sensor 206, the controller 205 may also control the oxygen source 210 and the air source 215 to discharge oxygen 212 and air 216 respectively, or to cease discharging oxygen 212 and air 216, as described below.

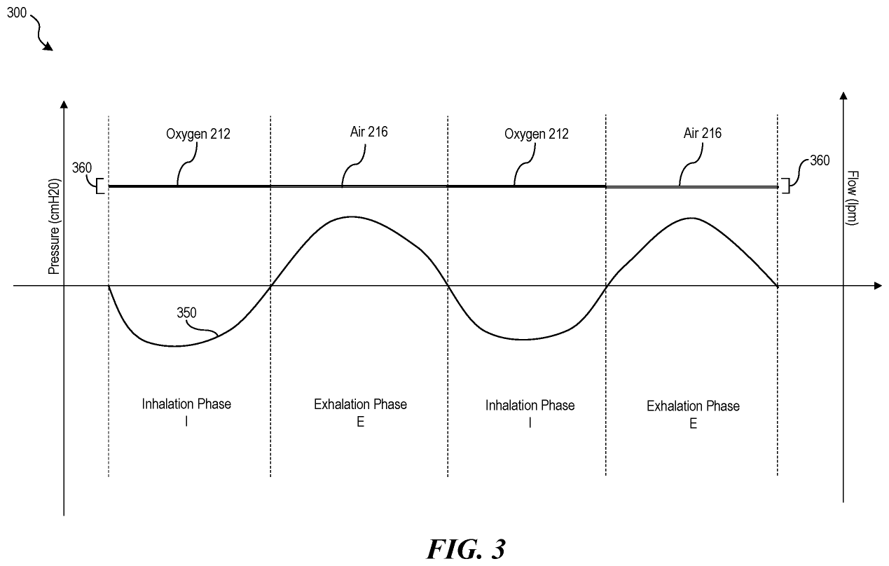

In operation, the system 200 delivers oxygen 212 during the patient's inhalation phase and air 216 during the patient's exhalation phase. FIG. 3, for example, is a graph illustrating various aspects of a high flow therapy administered to a patient using the system 200 in accordance with embodiments of the present technology. In particular, FIG. 3 illustrates two full breathing cycles for the patient P, each cycle having an inhalation phase I and an exhalation phase E. Line 350 illustrates a pressure within the system 200 in cmH₂O (e.g., as measured by the first pressure sensor 204 and/or the second pressure sensor 206). Line 360 illustrates a flow within the system 200 in lpm (e.g., as measured by the flow sensors 202).

The controller 205 can control the system 200 to deliver oxygen 212 during the inhalation phase I and air 216 during the exhalation phase E. For example, when the pressure within the system 200 (e.g., at the patient connection 235 and/or in the patient circuit 230) indicates the patient P is inhaling, the controller 205 can control the oxygen source 210 to discharge oxygen 216 at a target flow rate (e.g., a flow rate between 15 lpm and 60 lpm). When the pressure within the system 200 indicates the patient P is exhaling, the controller 205 can (1) direct the oxygen source 210 to cease delivering oxygen 212, and (2) direct the air source 215 to deliver air 216 at a target flow rate. As shown in FIG. 3, this can be repeated for any number of breath cycles such that the system 200 alternates between delivering oxygen 212 during the inhalation phase I and air 216 during the exhalation phase.

In some embodiments, the pressure changes from a negative value during inhalation to a positive value during exhalation. In such embodiments, detecting a change in pressure from a negative value to a positive value indicates that the patient P has transitioned from inhalation to exhalation, and detecting a change in pressure between a positive value and a negative value indicates that the patient P has transitioned from exhalation to inhalation. In other embodiments, the pressure changes from a first negative value during inhalation to a second negative value during exhalation, or from a first positive value during inhalation to a second positive value during exhalation. In such embodiments, the system 200 can be calibrated such that the controller 205 can nevertheless determine a transition between the inhalation phase I and the exhalation phase E, e.g., by the sensed pressure crossing a predetermined threshold value that indicates a transition between the inhalation phase I and the exhalation phase E.

As best shown by the line 360, the overall flow administered to the patient P during the inhalation phase I and the exhalation phase E can remain substantially constant, regardless of whether oxygen 212 or air 216 is being delivered. For example, the system 200 may administer oxygen 212 at 60 lpm during the inhalation phase I and administer air 216 at 60 lpm during the exhalation phase E. Accordingly, the flow provided to the patient P does not change (or at least does not substantially change) between the inhalation phase I and the exhalation phase E even through the type of gas being delivered changes. Of course, in some embodiments, the flow during inhalation and exhalation can be set such that flow during the inhalation phase I is different than (e.g., greater than or less than) flow during the exhalation phase E.

The system 200 therefore administers the oxygen 212 at high flows during patient inhalation, similar to standard high flow therapy systems. As a result, during inhalation, oxygen 212 is blown into the patient's lungs, fills the patient's alveoli, and provides an oxygenated breath to the patient P. Unlike standard high flow therapy systems, however, the air 216 is delivered to the patient during exhalation. As described above, the primary benefit of delivering gas to the patient during exhalation is to promote clearance of CO₂ that could otherwise be trapped in the patient's airways. However, it is not necessary to use oxygen to clear CO₂ since, during exhalation, the gas is not being used to provide an oxygenated breath to the user. Accordingly, the air 216 can promote clearance of CO₂ from the patient's airways as effectively as oxygen. The system 200 is therefore expected to use less oxygen than standard high flow therapy systems by virtue of administering the air 216 (e.g., instead of the oxygen 212) during patient exhalation. For example, in some embodiments the system 200 may use an amount of oxygen that is less than 75% of, less than 50% of, less than 40% of, less than 30% of, or less than 20% of the amount of oxygen used in standard high flow therapy. As a particular example provided for illustrative clarity only, a standard high flow therapy administering oxygen at 60 lpm for one minute uses 60 liters of oxygen. In contrast, administering high flow therapy as described herein at 60 lpm for one minute may use, for example, 20 liters of oxygen, with the remaining 40 liters comprising air administered during exhalation. In the foregoing example, the same high flow therapy (e.g., 60 lpm) is administered to the patient, but with only ⅓ of the oxygen usage.

In some embodiments, the system 200 may administer the oxygen 212 in combination with the air 216 during the inhalation phase I. For example, the system 200 may administer oxygen 212 for a first preset period at the beginning of the inhalation phase (e.g., 0.5 second, 0.75 second, 1 second, etc.) and then deliver air 216 to the patient for the remainder of the inhalation phase I. The oxygen 212 delivered to the patient P at the beginning of the inhalation phase I occupies the patient's lungs and provides the oxygenated breath to the user, whereas the air 216 provided at the end of the exhalation phase does not reach the patient's lungs and thus does not impact the FIO₂ value. This is expected to further reduce the amount of oxygen 212 used by the system 200 while maintaining the advantages of high flow therapy.

In some embodiments, the system 200 may administer the oxygen 212 in combination with the air 216 during the exhalation phase E. For example, the system 200 may administer the oxygen 212 at 30 lpm and the air 216 at 30 lpm such that flow administered is 60 lpm. Of course, the oxygen 212 and the air 216 can be administered at other ratios during exhalation, such as 3:1, 2:1, 1:2, 1:3, or other suitable ratios. In such embodiments, even though some oxygen is being delivered during exhalation, the oxygen consumption is still reduced relative to standard high flow therapy by replacing some of the oxygen typically delivered during exhalation with the air 216.

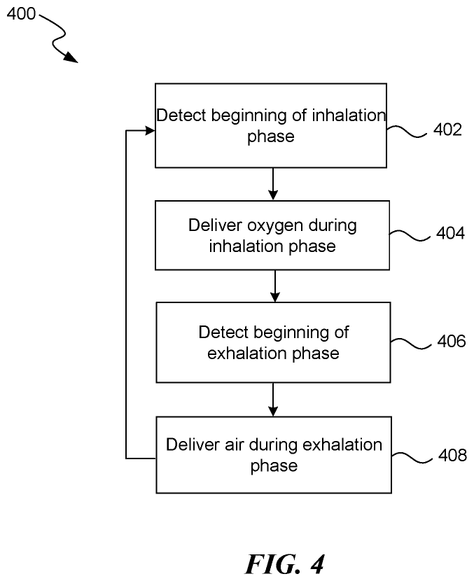

FIG. 4 is a flowchart of a method 400 of delivering high flow oxygen therapy to a patient in accordance with embodiments of the present technology. The method 400 can begin in block 402 by detecting a beginning of an inhalation phase of a breath. The beginning of the inhalation phase may be detected using one or more pressure sensors (e.g., the first pressure sensor 204 or the second pressure sensor 206 shown in FIG. 2) or via another suitable technique. In response to detecting the beginning of the inhalation phase, the method 400 can further include delivering oxygen to the patient during the inhalation phase. For example, the oxygen can be delivered to the patient at a flow rate of between 15 lpm and 60 lpm, such as between 30 lpm and 60 lpm, or between 45 lpm and 60 lpm, or another suitable high flow rate (e.g., such as adjusted for pediatric patients). This provides an oxygenated breath to the patient.

The method 400 can continue in block 406 by detecting the beginning of an exhalation phase of the breath. The beginning of the exhalation phase may be detected using one or more pressure sensors (e.g., the first pressure sensor 204 or the second pressure sensor 206 shown in FIG. 2) or via another suitable technique. In response to detecting the beginning of the exhalation phase, the method 400 can further include delivering air to the patient during the exhalation phase. For example, the air can be delivered to the patient at a flow rate of between 15 lpm and 60 lpm, such as between 30 lpm and 60 lpm, or between 45 lpm and 60 lpm, or another suitable high flow rate. This promotes clearance of CO₂ from the patient's airways. In some embodiments, the air can be delivered to the patient during the exhalation phase at the same or about the same flow rate as the oxygen delivered to the patient during inhalation phase. In other embodiments, the air can be delivered to the patient at a different flow rate than the oxygen. The operations in blocks 402-408 can be repeated for any number of cycles or for any duration to provide high flow therapy to the patient with reduced oxygen consumption relative to standard high flow oxygen therapy.

### EXAMPLES

Several aspects of the present technology are set forth in the following examples:
1. A high flow therapy system, comprising:
an oxygen source configured to provide oxygen to an oxygen delivery lumen at a first flow rate of between 15 lpm and 60 lpm;
an air source configured to provide air to an air delivery lumen at a second flow rate of between 15 lpm and 60 lpm; and
a controller configured to (i) direct the oxygen source to provide the oxygen to the oxygen delivery lumen at the first flow rate during an inhalation phase of a breath, and (ii) direct the air source to provide the air to the air delivery lumen at the second flow rate during an exhalation phase of the breath.
2. The system of example 1 wherein the controller is further configured to (iii) direct the oxygen source to cease delivery of the oxygen during the exhalation phase of the breath, and (iv) direct the air source to cease delivery of the air during the inhalation phase of the breath.
3. The system of example 1 wherein the controller is configured to direct the oxygen source to provide the oxygen to the oxygen delivery lumen during a first period of the inhalation phase, and wherein the controller is further configured to direct the air source to provide the air to the air delivery lumen during a second period of the inhalation phase.
4. The system of any of examples 1-3 wherein the first flow rate is between 30 lpm and 60 lpm, and wherein the second flow rate is between 30 lpm and 60 lpm.
5. The system of any of examples 1-4 wherein the first flow rate and the second flow rate are about the same.
6. The system of any of examples 1-5, further comprising a pressure sensor, wherein the controller is configured to identify the inhalation phase and/or the exhalation phase based at least in part on one or more inputs from the pressure sensor.
7. The system of any of examples 1-6, further comprising a first flow sensor configured to measure a flow of the oxygen from the oxygen source and a second flow sensor configured to measure a flow of air from the air source.
8. The system of any of examples 1-7, further comprising a housing, wherein the oxygen source and the air source are positioned within the housing.
9. The system of any of examples 1-8, further comprising a patient circuit including the oxygen delivery lumen and the air delivery lumen, wherein the oxygen delivery lumen and the air delivery lumen are fluidly isolated.
10. The system of example 9 wherein the oxygen delivery lumen is coupled to the air delivery lumen.
11. The system of any of examples 1-10, further comprising:
a first humidifier configured to heat and/or humidify the oxygen; and
a second humidifier configured to heat and/or humidify the air.
12. The system of example 11, further comprising a humidifier assembly, the humidifier assembly including the first humidifier and the second humidifier, and wherein the humidifier assembly is configured to isolate the oxygen and the air.
13. A method for delivering high flow therapy to a patient, the method comprising: detecting a beginning of an inhalation phase of a breath of the patient;
delivering oxygen to the patient during the inhalation phase at a first flow rate of between 15 lpm and 60 lpm;
detecting a beginning of an exhalation phase of the breath of the patient; and
delivering air to the patient during the exhalation phase at a second flow rate of between 15 lpm and 60 lpm.
14. The method of example 13, further comprising:
ceasing delivery of the air during the inhalation phase of the breath; and
ceasing delivery of the oxygen during the exhalation phase of the breath.
15. The method of example 13 or 14 wherein the first flow rate is between 30 lpm and 60 lpm, and wherein the second flow rate is between 30 lpm and 60 lpm.
16. The method of any of examples 13-15 wherein the first flow rate and the second flow rate are about the same.
17. The method of any of examples 13-16 wherein the oxygen is delivered through a first lumen and the air is delivered through a second lumen, wherein the first lumen is fluidly isolated from the second lumen along a substantial portion of the first lumen and the second lumen.
18. The method of any of examples 13-17, further comprising humidifying the oxygen before delivering the oxygen to the patient during the inhalation phase.
19. The method of any of examples 13-18, further comprising humidifying the air before delivering the air to the patient during the exhalation phase.
20. The method of any of examples 13-19 wherein delivering the oxygen to the patient during the inhalation phase delivers an oxygenated breath to the user, and wherein delivering the air to the to the patient during the exhalation phase clears carbon dioxide from the patient's airways.

### CONCLUSION

The systems and methods described herein can be implemented with and/or distributed across computing architecture. For example, many of the systems described herein include a memory storing data, software modules, instructions, or the like. The memories described herein can include one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory is a non-transitory computer-readable storage medium that stores, for example, programs, software, data, or the like.

As one of skill in the art will appreciate from the disclosure herein, various components of the systems described above can be omitted without deviating from the scope of the present technology. Likewise, additional components not explicitly described above may be added to the systems without deviating from the scope of the present technology. For example, it will be appreciated that specific embodiments of the technology have been described herein for purposes of illustration, but well-known structures and functions have not been shown or described in detail to avoid unnecessarily obscuring the description of the embodiments of the technology. Moreover, although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. Accordingly, the present technology is not limited to the configurations expressly identified herein, but rather encompasses variations and alterations of the described systems and methods.

Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Further, where specific integers are mentioned herein which have known equivalents in the art to which the embodiments relate, such known equivalents are deemed to be incorporated herein as if individually set forth.

1. A high flow therapy system, comprising:
an oxygen source configured to provide oxygen to an oxygen delivery lumen at a first flow rate of between 15 lpm and 60 lpm;
an air source configured to provide air to an air delivery lumen at a second flow rate of between 15 lpm and 60 lpm; and
a controller configured to (i) direct the oxygen source to provide the oxygen to the oxygen delivery lumen at the first flow rate during an inhalation phase of a breath, and (ii) direct the air source to provide the air to the air delivery lumen at the second flow rate during an exhalation phase of the breath.

2. The system of claim 1 wherein the controller is further configured to (iii) direct the oxygen source to cease delivery of the oxygen during the exhalation phase of the breath, and (iv) direct the air source to cease delivery of the air during the inhalation phase of the breath.

3. The system of claim 1 wherein the controller is configured to direct the oxygen source to provide the oxygen to the oxygen delivery lumen during a first period of the inhalation phase, and wherein the controller is further configured to direct the air source to provide the air to the air delivery lumen during a second period of the inhalation phase.

4. The system of claim 1 wherein the first flow rate is between 30 lpm and 60 lpm, and wherein the second flow rate is between 30 lpm and 60 lpm.

5. The system of claim 1 wherein the first flow rate and the second flow rate are about the same.

6. The system of claim 1, further comprising a pressure sensor, wherein the controller is configured to identify the inhalation phase and/or the exhalation phase based at least in part on one or more inputs from the pressure sensor.

7. The system of any of claim 1, further comprising a first flow sensor configured to measure a flow of the oxygen from the oxygen source and a second flow sensor configured to measure a flow of air from the air source.

8. The system of any of claim 1, further comprising a housing, wherein the oxygen source and the air source are positioned within the housing.

9. The system of any of claim 1, further comprising a patient circuit including the oxygen delivery lumen and the air delivery lumen, wherein the oxygen delivery lumen and the air delivery lumen are fluidly isolated.

10. The system of claim 9 wherein the oxygen delivery lumen is coupled to the air delivery lumen.

11. The system of claim 1, further comprising:
a first humidifier configured to heat and/or humidify the oxygen; and
a second humidifier configured to heat and/or humidify the air.

12. The system of claim 11, further comprising a humidifier assembly, the humidifier assembly including the first humidifier and the second humidifier, and wherein the humidifier assembly is configured to isolate the oxygen and the air.

13. A method for delivering high flow therapy to a patient, the method comprising:
detecting a beginning of an inhalation phase of a breath of the patient;
delivering oxygen to the patient during the inhalation phase at a first flow rate of between 15 lpm and 60 lpm;
detecting a beginning of an exhalation phase of the breath of the patient; and
delivering air to the patient during the exhalation phase at a second flow rate of between 15 lpm and 60 lpm.

14. The method of claim 13, further comprising:
ceasing delivery of the air during the inhalation phase of the breath; and
ceasing delivery of the oxygen during the exhalation phase of the breath.

15. The method of claim 13 wherein the first flow rate is between 30 lpm and 60 lpm, and wherein the second flow rate is between 30 lpm and 60 lpm.

16. The method of claim 13 wherein the first flow rate and the second flow rate are about the same.

17. The method of claim 13 wherein the oxygen is delivered through a first lumen and the air is delivered through a second lumen, wherein the first lumen is fluidly isolated from the second lumen along a substantial portion of the first lumen and the second lumen.

18. The method of claim 13, further comprising humidifying the oxygen before delivering the oxygen to the patient during the inhalation phase.

19. The method of claim 13, further comprising humidifying the air before delivering the air to the patient during the exhalation phase.

20. The method of claim 13 wherein delivering the oxygen to the patient during the inhalation phase delivers an oxygenated breath to the user, and wherein delivering the air to the to the patient during the exhalation phase clears carbon dioxide from the patient's airways.

21. The system of claim 1 wherein the oxygen is greater than 21% O₂.

22. The system of claim 1 wherein the oxygen is between about 80% and 100% O₂.

23. The system of claim 1 wherein the controller is further configured to (iii) direct the air source to provide the air to the air delivery lumen during the inhalation phase of the breath in combination with directing the oxygen source to provide the oxygen to the oxygen delivery lumen.

24. The method of claim 13 wherein the oxygen is greater than 21% O₂.

25. The method of claim 13 wherein the oxygen is between about 80% and 100% O₂.

26. The method of claim 13, further comprising delivering the air during the inhalation phase in combination with delivering the oxygen during the inhalation phase.
